(19) United States
(12) Patent Application Publication
(10) Pub. No.: US 2004/0028703 A1
(43) Pub. Date: Feb. 12, 2004

(54) Protein complex serving as a vehicle for orally administerable medicaments

(76) Inventors: Hans Bigalke, Hannover (DE); Jurgen Frevert, Berlin (DE)

Correspondence Address: GUDRUN E. HUCKETT, LONSSTR. 53, WUPPERTAL, 42289, DE

(21) Appl. No.: 10/333,477

(22) PCT Filed: Jul. 19, 2001
(86) PCT No.: PCT/DE01/02816

(30) Foreign Application Priority Data
Jul. 19, 2000 (DE) 10035155.5
Jul. 19, 2000 (DE) 10035156.7

(51) Int. Cl. A61K 39/395; A61K 39/08; A61K 38/19; C07K 14/33

(52) U.S. Cl. 424/239.1; 424/85.1; 424/178.1; 530/350; 530/351; 530/391.1

(57) ABSTRACT

The invention relates to a protein complex comprising one or more complex proteins or derivatives extracted from Clostridium botulinum of type A, B, C₁, C₂, D, E, F or G, and a selected polypeptide or low-molecular pharmacon.

The present invention relates to a protein complex comprising one or more complexing proteins or derivatives from Clostridium botulinum type A, B, C₁, C₂, D, E, F or G and a selected polypeptide or a pharmaceutical drug of a low molecular weight.

Due to the success brought about by biotechnological processes a number of highly effective pharmaceutical drugs has been developed, which drugs may, e.g., contain proteins as effective components. Apart from recombinant insulin, proteins of higher molecular weight such as growth factors, interleukins and monoclonal antibodies belong thereto. Some of these pharmaceutical drugs, e.g., erythropoetin (EPO), are the drugs having the biggest turnover. The number of proteinaceous pharmaceutical drugs will—last but not least also due to the knowledge derivable from the complete sequencing of the human genome—even increase in future times. All these novel pharmaceutical drugs exhibit a significant drawback as compared to convenient pharmaceutical drugs of low molecular weight: they are not resorbed orally. The drawbacks mentioned are likewise applicable for vaccines for an active immunization, e.g., tetanus toxoid.

The predominant number of drugs of low molecular weight may be administered orally. The substances traverse the mucosa of the intestine and enter into the blood circulation and are thus systemically available and reach the site where they exert their effects via the blood circulation system. This pathway is not available for proteinaceous pharmaceutical drugs, acid, labile drugs and drugs exhibiting an unfavorable charge. A number of mechanisms prevents the resorption of proteins. Beginning in the stomach, many proteins are denatured due to the low pH and lose their biological activity. Additionally, proteins are degraded by a number of pancreatic proteases (inter alia, trypsin, chymotrypsin, pepsin) into their amino acid residues which, in turn, can be resorbed. Even if a protein survived a proteolytic attack and reached the small intestine safely, it could not be readily resorbed since the intestinal wall is impermeable for substances of higher molecular weight to avoid an overflooding of the body of antigens. Furthermore, a number of pharmaceutical drugs exist that are not resorbed due to the unfavorable charge and hydrophobicity, respectively.

It is for these reasons that orally administered proteinaceous pharmaceutical drugs or vaccines and particular pharmaceutical drugs of low molecular do not exert any effect. They must be injected or, e.g., reach the site where they exert the effect via a nasal application.

A number of developments deals with the object to overcome the obstacles mentioned. In order to protect proteins and specific pharmaceutical drugs of low molecular weight from an inactivation and a degradation in the gastrointestinal tract one may encapsulate them into stomach-resistant capsules which will be resolved in the small intestine and release the pharmaceutically active protein or the pharmaceutical drugs of low molecular weight. This method suffers from the drawback that the protein and the pharmaceutical drugs of low molecular weight will not be degraded. However, these components will still not be able to penetrate the intestinal wall. Further developments attempt to benefit from carrier systems serving for an active transport of substances across the mucosa of the intestine, such as the carrier system of vitamin B. Taken alone, these methods are not successful but require additionally that the proteins and labile pharmaceutical drugs of low molecular weight are initially protected.

Accordingly, the object of the present invention is to provide means suitable to orally administer to a subject the desired polypeptide and pharmaceutical drugs of low molecular weight.

This object is solved by the subject matter as defined in the attached claims.

The present invention is further explained by the following figure.

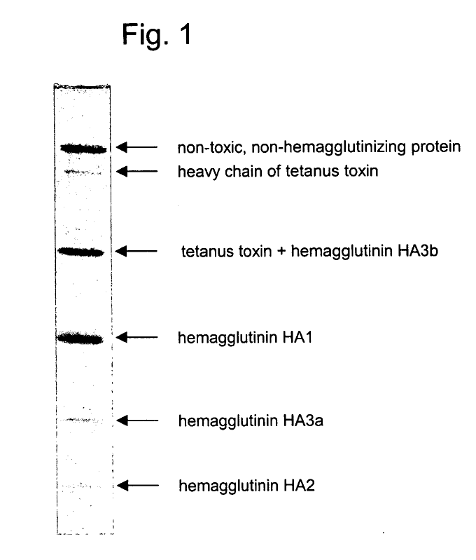

FIG. 1 schematically depicts the result of an SDS polyacrylamide gel electrophoreses (12%) of a protein complex with tetanus toxin according to the present invention.

The term “protein complex” as used herein defines a vehicle, by which further selected polypeptides are pharmaceutical drugs of low molecular weight can be transported into the human blood system and into the blood system of animals. The protein complex consists of at least one hemagglutinin and optionally non-toxic non-hemagglutinizing protein (NTHT) of the botulinum toxin complexes of at least one of the Clostridium botulinum types A, B, C₁, C₂, D, E, F or G.

The term “botulinum toxin complex” as used herein means a naturally occurring protein complex of the type A, B, C₁, C₂, D, E, F or G of Clostridium botulinum comprising the botulinum toxin, hemagglutinins and non-toxic non-hemagglutinizing protein (NTHT).

The term “polypeptide” or “selected polypeptide” as used herein means a peptide consisting of at least two amino acid residues. The polypeptide may be linear, circular or branched. Furthermore, the polypeptide may consist of more than one amino acid chain, wherein the chain may be connected to each other, e.g., via a disulfide bond. The polypeptides may further contain modified amino acid residues and the standard posttranslational modifications such as glycosylation. The polypeptides may be pharmacologically or immunologically active polypeptides or polypeptides used for diagnostic purposes, such as antibodies or ligands.

Bacteria of the strain Clostridium botulinum have found a route during evolution to introduce into the blood circulation of mammals via the gastrointestinal tract an intact protein—the Clostridium botulinum toxin.

Clostridium botulinum is classified into 8 serotypes which are distinct due to their toxins: type A, B, C₁, C₂, D, E, F and G. The proteins hereinafter frequently termed botulinum toxins, are proteins having a molecular weight of about 150 kDa. Botulinum toxin is usually taken up with contaminated food, is enterally resorbed and reaches the site to exert its effect, that is the motor endplate, where the nerve impulse is transferred to muscles. The toxins are taken up by the nerve cell and paralyze the secretion mechanism of acetylcholine in the nerve endings such that the muscle concerned will no longer be activated but relaxes.

However, botulinum toxin is not secreted in naked form from Clostridium botulinum but is produced in complexed form, that is, the clostridial cells produce not only the botulinum toxin but various further proteins that form—along with the toxin—a complex having a molecular weight of about 700 to about 900 kDa, the botulinum toxin complex. In various investigations it could be demonstrated that the formation of botulinum toxin complex is required for the oral toxicity of the botulinum toxin. It could be demonstrated that the botulinum toxin as present in the botulinum toxin complex exhibits an about 100,000-fold higher toxicity than the pure botulinum toxin. It is conceivable that the hemagglutinins are destined to attach the complex to the intestinal wall, thereby enabling the transport through the mucosa of the intestine into the blood circulation. Furthermore, it has been reported that the complex serves to protect the toxin against proteases in the gastrointestinal tract.

The other proteins (complexing proteins) are a number of hemagglutinins and a non-toxic non-hemagglutinizing protein (NTHT) exhibiting a molecular weight of about 120 kDa. For the botulinum toxin complex of type A were described the following hemagglutinins: Ha2 with about 16.9 kDa, Ha3a with about 21 kDa, Ha3b with about 52 kDa and Hal with about 35 kDa.

The complexes of the other toxin types B to G are constructed following a similar scheme. As an example, the complex of type B comprises, in addition to the NTHT, Ha-70 having a molecular weight of about 70 kDa, Ha-17 having a molecular weight of about 17 kDa and Ha-33 having a molecular weight of about 33 kDa (cf. Bhandari, M. et al., Current Microbiology 35, 207-214 (1997)).

In addition, East, A. K et al., System Appl. Microbiol. 17, 306-313 (1994)) describe the sequence of Ha-33 of type B as a comparison with the sequences of type A and type C. For type C and type D Ha-3b with a molecular weight of about 53 kDa and Ha3a with a molecular weight of about 22 to 24 kDa and Ha2 with a molecular weight of about 17 kDa (cf. Inoue, K. et al., Microbiology 145, 2533-2542 (1999)) have been described in addition to Ha-33 (=Ha1) having a molecular weight of about 33 kDa, which is likewise analogous to type A.

The complexes formed exhibit different compositions depending on their serotype, however. That is, a distinct number of the hemagglutinins and NTHT, respectively, are integrated into the complex. For the complex of type A the following composition has been calculated by, e.g., Inoue et al., Infection and Immunity 64 (5), 1589-1594 (1996)):

| protein | molar ratio |
|---|---|
| toxin | 1 |
| Ha-35 (=Ha1) | 7.76 |
| Ha-15 (=Ha2) | 2.71 |
| Ha-19 (=Ha3a) | 3.4 |
| Ha-52 (=Ha3b) | 2.24 |
| NTHT | 1.41 |

One aspect of the present invention is thus the provision of a protein complex comprising one or more complexing proteins or their derivatives of at least one of the Clostridium botulinum types A, B, C₁, C₂, D, E, F or G. The protein complex further comprises a selected polypeptide or a pharmaceutical drug of low molecular weight that is protected from degradation by proteases or acids in the gastrointestinal passage by the protein complex according to the invention when administered orally and is rendered systemically available by the complexing proteins, respectively. The selected polypeptide may be a pharmacologically active, an immunologically active polypeptide or a polypeptide used for diagnostic purposes. The selected pharmaceutical drug of low molecular weight may likewise be a pharmacologically active, an immunologically active pharmaceutical drug or a pharmaceutical drug used for diagnostic purposes, or any other medicament. The protein complex of the present invention is thus useful as a transport vehicle introducing the selected polypeptides and the pharmaceutical drugs of low molecular weight into the blood system of animals, preferably of mammals or birds, or preferably of humans, and thus transporting them to the site of their effect. A further aspect of the present invention is thus to provide a protein complex as a therapeutic agent, vaccine or diagnostic agent, for human and/or veterinary medicine. A further aspect of the further invention is the use of a protein complex comprising one or more complexing proteins of at least one of the Clostridium botulinum type A, B, C₁, C₂, D, E, F or G as a transport vehicle for pharmacologically active polypeptides or substances (pharmaceutical drugs) of low molecular weight, immunologically active polypeptides or substances (pharmaceutical drugs) of low molecular weight or polypeptides or substances (pharmaceutical drugs or diagnostic drugs) of low molecular weight for diagnostic purposes.

The protein complex consists of hemagglutinins and NTHT and may be equivalent to the naturally occurring complexes of type A, B, C₁, C₂, D, E, F or G of Clostridium botulinum. The protein complex may, however, exhibit a composition other than its natural composition. For example, it may consist only of hemagglutinin without the NTHT proteins. Furthermore, the protein complex may consist of less types of hemagglutinins than the naturally occurring complex, preferably of three different types of hemagglutinins, preferably of two and more preferably of only one type of hemagglutinin, wherein the protein complex may comprise the NTHT protein, or may not comprise this protein. The protein complex may further consist of a mixture of one or more types of hemagglutinin and/or NTHT proteins of the different serotypes.

Preferred are protein complexes corresponding to the naturally occurring protein complexes from Clostridium botulinum of type A, B, C₁, C₂, D, E, F or G, for example a protein complex with Ha1, Ha2, Ha3a, Ha3b and NTNH of Clostridium botulinum type B. The protein complex may additionally be composed of Ha1, Ha2, Ha3a and NTNH, of Ha1, Ha2, Ha3b and NTNH, of Hal and Ha3a, Ha3b and NTNH, of Ha2, Ha3a, Ha3b and NTNH, of Hal, Ha2 and NTNH, of Hal, Ha3a and NTNH, of Ha1, Ha3b and NTNH, of Ha2, Ha3a and NTNH, of Ha2, Ha3b and NTNH, of Ha3a, Ha3b and NTNH, or of further arbitrary combinations of the complexing proteins listed. The protein complex may further be composed of one of the hemagglutinins and NTNH. In addition, the protein complex may be composed of the combinations of hemagglutinins given above without NTNH. According to the exemplary protein complexes of type B, further preferred protein complexes are those consisting of the hemagglutinins and/or NTNH of type A, C₁, C₂, D, E, F or G.

Further preferred are the protein complexes according to the present invention, wherein one ore more complexing proteins are bound to the selected polypeptide or the pharmaceutical drugs of low molecular weight via a chemical bond. This bond could be cleaved subsequent to resorption in the blood such that the polypeptide or the medicament of low molecular weight may reach its site to exert its effect. The selected polypeptide or the pharmaceutical drug of low molecular weight may be bound to the complexing proteins via a cross-linking agent. Preferred crosslinking agents are, e.g., N-(4-azidophenylthio)phthalimide, 4,4′-dithiobis-phenylazido, dithiobispropionimidate, 3,3′-dithiobis(sulphosuccinimide-propionate), ethyl-4-azidophenyl-1,4-dithiopropionate, N-sulphosuccinidyl-(4-azidophenyl)-1,3′-dithiopropionate, sulphosuccinidyl-2-(p-azidosalicylamine)-ethyl-1,3′-dithiopropionate, N-succinimide-3-(2-pyridyldithio)propionate or bis-(2-(succinimidyloxycarbonyloxy)-ethyl)sulphone. Preferred is a single complexing protein connected to the selected polypeptide or the pharmaceutical drug of low molecular weight via a chemical bond.

A further aspect of the present invention is the provision of a method for the preparation of the protein complex of the invention. The method comprises the following steps:

a) isolation of at least one botulinum toxin complex of type A, B, C₁, C₂, D, E, F or G from Clostridium botulinum at a pH of 2.0 to 6.5,

b) increase of the pH to 7.0 to 10.0,

c) separation of the botulinum toxin from the complexing proteins by means of chromatographic procedures,

d) mixing the complexing proteins obtained in step c) with a selected polypeptide or a pharmaceutical drug of low molecular weight, or

d′) separation of the complexing proteins obtained in step c) and mixing at least one complexing protein with a selected polypeptide or a pharmaceutical drug of low molecular weight, and

e) dialysis of the mixture from step d) or d′) against a buffer at a pH of 2.0 to 6.5, and optionally

f) coupling of the complexing proteins with the selected polypeptide or the pharmaceutical drug of low molecular weight via a chemical bond.

Preferred is a method wherein the at least two complexing proteins mixed in steps d) or d′) are derived from a single or from different botulinum toxin complex types.

The complexing proteins may be isolated from the natural botulinum toxin complexes. An exemplary method for their isolation is as follows: First the botulinum toxin complex of clostridial cells is isolated at acid pH, preferably at a pH of 2.0 to 6.5, more preferably at pH 4.0 to 6.5, still more preferably at pH 6.0. After an increase of the pH to 7.0 to 10.0, preferably to 7.0 to 8.0, the botulinum toxin will be separated by means of chromatographic procedures. This procedure can be performed since the complex is stabile at a pH of <6.5 and decomposes at neutral and alkaline pH and releases the toxin. Another polypeptide to be orally administered may subsequently be added to the toxin-free complexing proteins. The pH will be decreased by dialysis against a buffer that is conventional in protein chemistry, in particular against a phosphate, acetate or citrate buffer at a pH of 2.0 to 6.5, preferably 4.0 to 6.0, more preferably at pH 5.5. During these steps, a new complex is formed which guarantees the oral bioavailability of the polypeptide bound.

Further chromatographic procedures, procedures to concentrate and precipitations which are standard in protein chemistry, may also be used for the isolation of the complexing proteins.

The complexing proteins may be produced by means of DNA recombination techniques in specific host organisms as the DNA sequences are known. The complexing proteins thus produced may exhibit further modifications, that is, they may be derivatives of the complexing proteins. Modifications do not only mean deletions, additions, insertions or substitutions but include also chemical modifications of amino acids, e.g. methylations or acetylations as well as posttranslational modifications, e.g., glycosylations or phosphorylations. The expression of desired proteins in different hosts belongs to the state of the art of the person of average skill and needs no further description. The complexing protein required for the formation of the protein complex may be expressed in a host organism separately or simultaneously. Preferred is the production of the recombinant complexing proteins in bacteria, e.g., in E. coli, Bacillus subtilis and/or Clostridium difficile, or in eukaryotic cells, e.g., in CHO cells, in insect cells, e.g., by means of the baculovirus system, or in yeast cells. The complexing proteins may be isolated and added to the selected polypeptide or the pharmaceutical drug of low molecular weight according to the procedure described above. Furthermore, the selected polypeptide may be expressed in the host organism simultaneously with the complexing protein. Particularly preferred is the simultaneous or separate production of the respective complexing proteins along with the selected polypeptide via a YAC in yeast.

The protein complexes according the present invention may further consist of a mixture of recombinantly produced complexing proteins and complexing proteins isolated from natural botulinum toxin complexes.

The pharmacologically or immunologically active polypeptides which can be orally administered by means of the protein complex according to the invention may be any therapeutically or prophylactically effective polypeptides that had previously to be administered parenterally. The polypeptides may be, e.g., hormones, cytokines, enzymes, growth factors, antigens, antibodies, inhibitors, receptor agonists or antagonists, or blood clotting factors. It does not matter whether the polypeptides have been prepared recombinantly or isolated from their natural sources. Preferred polypeptides are insulin, erythropoetin, interferons, interleukins, HIV protease inhibitors, GM-CSF (granulocyte/macrophage stimulating factor), NGF (nerve growth factor), PDGF (platelet derived growth factor), FGF (fibroblast growth factor), plasminogen-activators, e.g., t-PA (tissue plasminogen activator), renin inhibitors, human growth factor, IGF (insulin-like growth factor), vaccines such as tetanus vaccine, hepatitis B vaccine, diphtheria vaccine, antibodies such as herceptin (antibody against Her2), antibodies against TNF (tumor necrosis factor), calcitonin, urokinase, streptokinase, inhibitors of angiogenesis, factor VIII, factor Xa antagonists, metalloprotease inhibitors.

The polypeptides used for diagnostic purposes can be, e.g., antibodies or ligands, wherein the polypeptides may exhibit a label. The label may be any label that is detectable in the body of humans or animals. Preferred labels are isotopes, e.g., ¹³C or radioactive labels. The labeled antibodies may be used to detect tumors, the labeled ligands may be used to detect, e.g., pathologic receptors.

The pharmaceutical drugs of low molecular weight that are made bioavailable can, e.g., be neomycin, salbutamol, pyrimethamin, methicillin, pethidin, ketamin or mephenesin.

The following examples explain the invention in more detail and should not be construed to limit the present invention.

### EXAMPLES

### Example 1

### Preparation of the Complexing Proteins from C. botulinum Type B

C. botulinum type B was fermented in a 20-L-fermenter according to published methods (cf. Evans et al., Eur. J. Biochem. 154, 409-416 (1986)). The fermentation medium comprises 2% proteose peptone no. 2 (DIFCO), 1% yeast extract, 1% glucose and 0.05% sodium thioglycolate. After growth for 72 h at 33° C. the toxic complex was precipitated by addition of 3 N H₂SO₄. The precipitate was extracted twice with 250 ml 0.2 M Na-phosphate pH 6.0. Nucleic acids were precipitated from the combined extracts by addition of 125 ml 2% protamine sulfate. Subsequently, the toxic complex was precipitated by means of 233 g ammonium sulfate (14 h at 2-8° C.). the precipitate was dissolved in 125 ml 50 mM Tris/HCl, 1 mM EDTA and dialyzed against this buffer at 2-8° C. overnight (2×2 l). Undissolved particles were separated via centrifugation (15 min, 15,000 rpm). 429 mg protein thus obtained were chromatographed through a Sepharose Q column (2.6×25 cm). Bound protein was eluted with a NaCl gradient (0-500 mM). The free neurotoxin of type B was eluted at about 100 mM NaCl, the complex was released at about 250 mM NaCl. The chromatography resulted in a yield of 151 mg protein.

### Example 2

### Separation of Contaminants of Botulinum Toxin Type B from Complexing Proteins

33 mg of the complexing proteins (pooled fractions following Sepharose Q chromatography) still contaminated with botulinum toxin were dialyzed against 50 mM Tris/HCl pH 7.9, 2 mM EDTA (2×1 l) overnight. The protein solution was chromatographed through a Q Hyper-D column (2.6×8 cm) and bound protein was eluted with a NaCl gradient (0-0.400 mM). The neurotoxin was released at a NaCl concentration of about 100 mM, the complexing proteins appeared at about 190 mM NaCl. In SDS-PAGE the portion of the neurotoxin was <1% of the analyzed proteins.

### Example 3

### Separation of Traces of Neurotoxin by Means of Affinity Chromatography for the Isolation of the Protein Complex (apo Complex).

In order to purify the complexing proteins from traces of neurotoxin an affinity chromatography was performed. Rabbits were immunized with detoxified homogeneous neurotoxin. The antisera obtained were purified by means of ammonium sulfate precipitation. The neurotoxin-specific antibodies could be purified via an affinity chromatography. For this purpose, 3 mg of the pure neurotoxin were immobilized on 0.6 g re-hydrated CnBr-Sepharose (following the recipe of the manufacturer). Antiserum (following ammonium sulfate precipitation) specific for the neurotoxin type B were chromatographed through a column (0.5×3 cm) which was filled with the synthesized matrix following dialysis against 20 mM sodium phosphate pH 7.0, 0.5 M NaCl. The toxin-specific antibodies were obtained by elution with 0.1 M glycine pH 2.7 (yield: 1.57 mg). 1.25 mg of the purified neurotoxin antibodies were immobilized on 1 g CNBr-Sepharose. Subsequently, 11.6 mg of the complex (following the Q Hyper-D chromatography) in 50 mM Tris/HCl pH 7.9, 2 mM EDTA pH 7.9 were chromatographed over this antibody affinity column. The solution was circulated repeatedly through the column overnight (16 h), with a flow rate of 40 ml/h. Bound neurotoxin-containing complex could be released with 0.1 M glycine pH 2.7. In the affinity-purified complex (9.8 mg) no neurotoxin could be detected any longer in a biological detection assay (phrenic test/assay: Goeschel et al., Experimental Neurology 147, 96-102 (1987)).

### Example 4

### Formation of a Protein Complex with Tetanus Toxin According to the Present Invention

(A) 200 μg pure tetanus toxin were added to 1 mg of the purified complexing proteins in 1 ml 50 mM Tris/HCl-buffer, pH 8.0. Subsequently, it was dialyzed against 50 mM citrate/phosphate-buffer pH 6.0 overnight. An aliquot (25 μl) was analyzed in a 50 mM Na-citrate buffer on a gel filtration column (Bioselect SEC 250-5). A single peak appeared, the peak corresponding to a molecular weight of about 500 kDa. The peak fraction was subjected to an SDS-PAGE. Both the bands of the complexing proteins and of the tetanus toxin could be detected. Accordingly, a novel protein complex with the heterologous toxin had been formed.

(B) 6 mg tetanus toxin and 6 mg apo complex (see Example 3) in 3 ml Tris/HCl, pH 7.9, 2 mM EDTA were dialyzed against 50 mM sodium phosphate, 250 mM NaCl, 2 mM EDTA, pH 7.0 for two days at 2-8° C. and subsequently dialyzed against the same buffer but at pH 6.0 for five days. Afterwards, 346 μl 4 M ammonium sulfate (0.75 M) were added to 1.5 ml of this solution, thereby precipitating the complex. The pellet was dissolved in 50 mM sodium phosphate, 150 mM NaCl, 2 mM EDTA, pH 5.9, and an aliquot thereof was analyzed via gel filtration. For this purpose a Biosep SEC 3000 7.8×300 mM (Phenomenex) was used (flow rate 0.5 ml/min). >90% of the protein were eluted in a high molecular weight peak (Mr>500000). The analysis of the peak fraction in 12% SDS-PAGE demonstrated that the protein complex comprised tetanus toxin. The presence of tetanus toxin was confirmed in the phrenic assay.

### Example 5

### Assay with the Tetanus Toxin-Protein Complex In Vivo in Mice

1 mg tetanus toxin was added to 5 mg of the purified complexing proteins in 2.5 ml 50 mM Tris/HCl, pH 8.0. Subsequently, it was dialyzed against 50 mM citrate phosphate buffer pH 6.0 overnight. 25 μl of the solution were analyzed for presence of tetanus toxin in the protein complex (see Example 4 (A)). 0.5 ml each were administered to 5 CD1 mice via pharyngeal tube/probe. 3 further mice (control) were treated with an equivalent amount of tetanus toxin. The mice treated with tetanus toxin-protein complex died from tetanus 24 h later, whereas the control mice did not exhibit any indication of tetanus.

### Example 6

### Assay with the Tetanus Toxin-Protein Complex In Vivo in Rats

5 Wistar rats (180-200 g) were each treated with 2 μg of the protein complex of the present invention (see Example 4 (B)) in 0.5 ml sodium phosphate, 150 mM NaCl, 2 mM EDTA, 100 μg BSA/ml, pH 6.0 via pharyngeal tube/probe. 3 further rats (control) were treated with an equivalent amount of tetanus toxin in the same buffer. The rats treated with tetanus toxin-protein complex died from tetanus within 24 h, whereas the control rats did not exhibit any indication of tetanus.

### Example 7

### Formation of a Protein Complex According to the Present Invention with Insulin

(A) 10 mg of the purified complexing proteins were dialyzed overnight with 0.5 mg insulin in a 50 mM citrate/phosphate buffer. A sample thereof was analyzed for complex formation in gel filtration. It appeared a peak corresponding to a molecular weight of >500 kDa. An aliquot of the peak fraction was analyzed in an SDS-PAGE. The peak fraction contained both the bands of the complexing proteins as well as the band of insulin.

(B) 3 mg of the purified complexing proteins were dialyzed with 0.5 mg insulin in a 50 mM phosphate buffer pH 7.0 for two days, followed by a dialysis against 50 mM phosphate, pH 6.0 for five days. Subsequently, ammonium sulfate precipitation was performed again. A sample was analyzed for complex formation by means of gel filtration. It appeared a peak corresponding to a molecular weight of >500 kDa. An aliquot of the peak fraction was analyzed in an SDS-PAGE. The peak fraction contained both the bands of the complexing proteins as well as the band of insulin.

### Example 8

### Glucose Stress Test with Mice

After the blood sugar level had been determined 1 ml of a 10% saccharose solution was administered to 10 CD1 mice via pharyngeal tube/probe. Each 1 mg of an insulin-protein complex was administered to 5 mice via pharyngeal tube/probe. In 30-min intervals the blood sugar level of the mice was determined. The result was that the blood sugar level of the treated mice was 25 to 40% below the average blood sugar level of the untreated mice.

### Example 9

### Glucose Stress Test with Rats

After the blood sugar level had been determined 1 ml of a 10% saccharose solution was administered to 6 Wistar rats via pharyngeal tube/probe. Each 0.5 mg of an insulin-protein complex was administered to 3 rats via pharyngeal tube/probe. In 30-min-intervals the blood sugar level of the rats was determined. The result was that the blood sugar level of the treated rats was 25 to 40% below the average blood sugar level of the untreated rats.

### Example 10

### Oral Immunization Against Tetanus

(A) 30 mg of a preparation of complexing protein were added 3 mg tetanus toxoid (mutated tetanus toxin). The mixture was dialyzed overnight against 50 mM citrate/phosphate buffer pH 5.5. 1 mg tetanus toxoid-protein complex each was administered to 5 CD1-mice via pharyngeal tube/probe. After two and six weeks the same dose was administered. Two weeks after the last treatment blood was taken and the antibody titer determined by means of ELISA. The mice had developed an antibody titer against the toxin (>1:1000), which is in contrast to five control mice which had obtained the same dose of toxoid not bound to the complex. It could furthermore be shown in a neutralization assay that the sera inactivated the activity of the toxin.

(B) 10 mg of a preparation of complexing protein were added 3 mg tetanus toxoid (mutated recombinant tetanus toxin). The mixture was dialyzed two days against 50 mM phosphate buffer pH 7.0 and subsequently three days at pH 6.0. 0.5 mg tetanus toxoid-complex each was administered to 5 CD1-mice via pharyngeal tube/probe. After two and six weeks the same dose was administered. Two weeks after the last treatment blood was taken and the antibody titer determined by means of ELISA. The mice had developed an antibody titer against the toxin (>1:1000), which is in contrast to five control mice which had obtained the same dose of toxoid not bound to the complex. It could furthermore be shown in a neutralization assay that the sera inactivated the activity of the toxin.

### Example 11

### Preparation of a Complex with Recombinant Complexing Proteins of Clostridium botulinum Type A

In order to prepare a recombinant complex the distinct protein components were prepared in E. coli (cf. Fujinaga, Y. et al., FEBS Letters 467, 179-183 (2000)). The method is analogous to the preparation of the hemagglutinins ((HA 1: Mr about 33 kDa, HA 2: Mr about 17 kDa, HA 3a: Mr about 21 kDa, HA 3b: Mr about 48 kDa) in E. coli in a pGEX-SX-3 expression vector as GST fusion proteins. After purification through a Glutathion-Sepharose 4B column the glutathion-S transferase was cleaved off by means of factor Xa. After separation of factor Xa and GST the pure recombinant proteins were isolated. Following the same method, the non-toxic, non-hemagglutinizing complexing protein has been prepared. The recombinant complexing proteins were dialyzed against a 50 mM Tris/HCl buffer pH 8.0 overnight (protein concentration 1-1.5 mg/ml).

In order to prepare a complex with tetanus toxin the components were mixed in the following molar ratios:

| | molar ratio | μg |
|---|---|---|
| Ha1 | 8 | 264 |
| Ha2 | 3 | 51 |
| Ha3a | 3 | 63 |
| Ha3b | 3 | 144 |
| tetanus toxin | 1 | 150 |

The protein mixture was dialyzed against a 50 mM sodium citrate buffer pH 5.5 for 16 h. A sample of 25 μl was analyzed for complex formation by means of gelfiltration. The protein appears in a peak corresponding to a molecular weight of about 500 kDa. The analysis of the peak fraction in an SDS-PAGE resulted not only in the complexing proteins but also in the bands of the tetanus toxin (150 kDa).

### Example 12

### Assay with a Recombinant Complex with Mice

The complex described in Example 10 (A) was tested with three CD1 mice. 50 μg of the recombinant complex was administered to the mice via a pharyngeal Tube/probe. All three mice died from tetanus within 48 h, whereas three mice which had been administered with an equivalent amount of pure tetanus toxin (11 μg) showed no indication of tetanus.

1. A protein complex comprising one or more complexing proteins from at least one of type A, B, C₁, C₂, D, E, F or G of Clostridium botulinum and a selected polypeptide or a pharmaceutical drug of low molecular weight, wherein the selected polypeptide is not a botulinum toxin.

2. The protein complex of claim 1, wherein the complexing proteins are a mixture of complexing proteins of at least one of type A, B, C₁, C₂, D, E, F or G of Clostridium botulinum.

3. The protein complex of claim 1 or 2, wherein the selected polypeptide is a pharmacologically active, an immunologically active polypeptide or a polypeptide used for diagnostic purposes.

4. The protein complex of claim 3, wherein the pharmacologically or immunologically active polypeptide is a hormone, a cytokine, an enzyme, a growth factor, an antigen, an antibody, an inhibitor, a receptor agonist or antagonist or a blood clotting factor.

5. The protein complex of claim 3, wherein the polypeptide used for diagnostic purposes is a labeled antibody or a labeled ligand.

6. The protein complex of claim 1 or 2, wherein the pharmaceutical drug of low molecular weight is neomycin, salbutamol, pyrimethamin, methicillin, pethidin, ketamin or mephenesin.

7. The protein complex of any of claims 1 to 6, wherein a complexing protein is bound to the selected polypeptide or with the pharmaceutical drug of low molecular weight via a chemical bond.

8. The protein complex of any of claims 1 to 7 as a therapeutic agent, a vaccine or a diagnostic agent in human and/or veterinary medicine.

9. Method for the preparation of the protein complex of any of claims 1 to 7, the method comprising the following steps:
a) isolation of at least one botulinum toxin complex of type A, B, C₁, C₂, D, E, F or G from Clostridium botulinum at a pH of 2.0 to 6.5,
b) increase of the pH to 7.0 to 10.0,
c) separation of the botulinum toxin from the complexing proteins by means of chromatographic procedures,
d) mixing the complexing proteins obtained in step c) with a selected polypeptide or a pharmaceutical drug of low molecular weight, or
d′) separation of the complexing proteins obtained in step c) and mixing at least one complexing protein with a selected polypeptide or a pharmaceutical drug of low molecular weight, and
e) dialysis of the mixture from step d) or d′) against a buffer at a pH of 2.0 to 6.5, and optionally
f) coupling of the complexing proteins with the selected polypeptide or the pharmaceutical drug of low molecular weight via a chemical bond.

10. The method of claim 9, wherein the at least two complexing proteins mixed in steps d) or d′) are derived from a single or several different types of botulinum toxin complexes.

11. Method for the preparation of the protein complex of any of claims 1 to 7, wherein the complexing proteins are produced by means of recombinant DNA techniques.

12. Use of a protein complex comprising one or more complexing proteins of at least one of type A, B, C₁, C₂, D, E, F oder G of Clostridium botulinum as a transport vehicle for pharmacologically active polypeptides or substances of low molecular weight, immunologically active polypeptides or substances of low molecular weight, or polypeptides for diagnostic purposes or substances of low molecular weight.
